(19) United States
(12) Patent Application Publication
(10) Pub. No.: US 2007/0083192 A1
(43) Pub. Date: Apr. 12, 2007

(54) Apparatus and method for ablation of targeted tissue

(76) Inventor: Eric Welch, Miramar, FL (US)

Correspondence Address: FAY KAPLUN & MARCIN, LLP, 15O BROADWAY, SUITE 702, NEW YORK, NY, 10038, US

(21) Appl. No.: 11/245,575

(22) Filed: Oct. 7, 2005

(51) Int. Cl. A61B 18/14

(52) U.S. Cl. 606/41

(57) ABSTRACT

A tissue treatment device comprises an elongate member including a lumen extending therethrough and an inflatable member mounted to the distal portion of the elongated member and surrounding an opening of the lumen, an outer surface of the inflatable member including a conductive region having a first degree of energy conductivity and a non-conductive region having a second degree of energy conductivity lower than the first degree of energy conductivity and an energy source supplying energy to the inflatable member to transfer energy from the conductive region to target tissue. A method of ablating tissue comprises lifting the target mucosal tissue from underlying tissue and inserting a distal end of an endoscope to a desired position relative to the target mucosal tissue in combination with inserting an elongated member including an inflatable member mounted thereto through the endoscope to the desired position, the inflatable member including a conductive region having a first degree of energy conductivity and a non-conductive region having a second degree of energy conductivity, the second degree of energy conductivity being lower than the first degree of energy conductivity, inflating the inflatable member to place the conductive region in contact with the target mucosal tissue and supplying energy to the inflatable member to transfer energy from the conductive region to the target mucosal tissue.

### BACKGROUND

Endoscopic procedures to treat disorders of the gastro-intestinal (“GI”) canal, of the vascular system, the urinary tract and other body lumens are becoming increasingly common. Most often endoscopes are used to provide a conduit permitting users to insert various medical devices therethrough to internal locations requiring treatment. Once the inserted device has reached the tissue to be treated, it is manipulated using controls which remain outside the body.

When medications have proved insufficient, the treatment of gastro-esophageal reflux disease (GERD) relies in large part on endoscopic procedures. For example, in the Stretta procedure, radio frequency (RF) energy is used to heat and shrink the lower esophageal sphincter and surrounding tissues to form a more secure valve. However, it is difficult to apply the RF energy only to the target locations without damaging surrounding tissues.

Lesions of the GI tract or other hollow organs are also treated with RF energy. For example, in endoscopic mucosal resection procedures, target tissue is removed from underlying tissue by lifting the target tissue and cutting this target tissue from the surrounding tissue. It has also proven difficult to employ RF energy to cut this target tissue from the surrounding tissue without damaging non-targeted healthy tissues.

### SUMMARY OF THE INVENTION

In one aspect, the present invention is directed to a tissue treatment device comprising an elongate member including a lumen extending therethrough to an opening formed in a distal portion thereof and an inflatable member mounted to the distal portion of the elongated member and surrounding the opening, an outer surface of the inflatable member including a conductive region having a first degree of energy conductivity and a non-conductive region having a second degree of energy conductivity, the second degree of energy conductivity being lower than the first degree of energy conductivity in combination with an energy source for supplying energy to the inflatable member to transfer energy from the conductive region to target tissue.

The present invention is further directed to a method of resecting target mucosal tissue within a body lumen comprising lifting the target mucosal tissue from underlying tissue and inserting a distal end of an endoscope to a desired position relative to the target mucosal tissue in combination with inserting an elongated member including an inflatable member mounted thereto through the endoscope to the desired position, the inflatable member including a conductive region having a first degree of energy conductivity and a non-conductive region having a second degree of energy conductivity, the second degree of energy conductivity being lower than the first degree of energy conductivity, inflating the inflatable member to place the conductive region in contact with the target mucosal tissue and supplying energy to the inflatable member to transfer energy from the conductive region to the target mucosal tissue.

### BRIEF DESCRIPTION OF THE DRAWINGS

FIG. 1 is a schematic diagram showing a first embodiment of a device according to the present invention;

FIG. 2 is a schematic diagram showing a device according to a second embodiment of the present invention; and

FIG. 3 is a schematic diagram showing a device according to a third embodiment of the present invention.

FIG. 4 is a schematic diagram showing a device according to a fourth embodiment of the present invention.

FIG. 5 is a schematic diagram showing a device according to a fifth embodiment of the present invention.

FIG. 6 is a schematic diagram showing a device according to a sixth embodiment of the present invention.

FIG. 7 is a schematic diagram showing a device according to a seventh embodiment of the present invention.

FIG. 8 is a schematic diagram showing a device according to a eighth embodiment of the present invention.

FIG. 9 is a schematic diagram showing a device according to a ninth embodiment of the present invention.

FIG. 10 is a schematic diagram showing a device according to a tenth embodiment of the present invention.

FIG. 11 is a schematic diagram showing a device according to a eleventh embodiment of the present invention.

FIG. 12 is a schematic diagram showing a device according to a twelfth embodiment of the present invention.

FIG. 13 is a schematic diagram showing a device according to a thirteenth embodiment of the present invention.

FIG. 14 is a schematic diagram showing a device according to a fourteenth embodiment of the present invention.

FIG. 15 is a schematic diagram showing a device according to a fifteenth embodiment of the present invention.

### DETAILED DESCRIPTION

The present invention may be further understood with reference to the following description and the appended drawings, wherein like elements are referred to with the same reference numerals. The present invention is related to medical devices utilizing RF energy to ablate target portions of tissue within hollow organs.

Endoscopic mucosal resection (EMR) is a medical procedure used to obtain a tissue sample or to resect a lesion. During EMR procedures, a lesion or other portion of target tissue is separated from an underlying tissue layer (e.g., muscularis propria) by lifting it away from the underlying tissue on a layer of fluid. For example, saline solution or another more viscous fluid may be injected submucosally to lift the lesion or target tissue. Various cutting, snaring or suction techniques may then be used to resect the target tissue and to remove it from the site. These techniques are particularly well suited to treat tissues in the esophagus, the colon and other regions of the GI tract.

The above described procedures, as well as other resection and ablation techniques rely on accurate placement of cutting surfaces of the resection device. In particular, when RF energy is used to sever tissue, it is important to accurately place the electrodes so that non-targeted tissue is not damaged. This task is especially difficult when manipulating a device including electrodes at the distal end of an endoscope. The ability of the surgeon to observe the target tissue and the electrodes and to accurately place the electrodes on the target tissue is also limited. by the relatively small field of view provided by the vision elements of the endoscope.

The methods and devices according to exemplary embodiments of the present invention allow for greater precision in the ablation, cauterization and/or resection of target tissues within hollow organs. In particular, the features the present invention is well suited for endoscopic mucosal resections of the GI tract and other RF ablation procedures such as the Stretta™ procedure to treat GERD. In addition to the GI tract, the embodiments of the present invention are useful in the treatment of other hollow organs such as the urological tract, the vascular system etc.

According to the invention, the cutting, the cauterization and/or ablation of target tissue is carried out by an inflatable member having one or more conductive patterns formed on its surface. Those skilled in the art will understand that the same apparatus described below may be used to carry out other therapeutic tissue treatments by varying the frequency, level and/or waveform of the energy applied. The patterns may be formed in any of a variety of shapes desired for the particular application ranging from simple concentric bands to complex geometric shapes. As would be understood by those skilled in the art, the complex conductive patterns may be designed to affect specific portions of target tissue without damaging surrounding non-targeted tissue. After the inflatable member has been placed within the hollow organ adjacent to the target tissue, it is inflated so that its outer surface presses against the surrounding target tissue around the entire circumference of the hollow organ. Various visualization methods may be used to verify the correct positioning of the inflatable member. RF energy then supplied to the inflatable member to flow through the conductive region(s) thereof to apply energy to the tissue in contact with these conductive regions. Because the shape and size of the region of targeted tissue is ablated without moving the device (i.e., based on the pattern of the conductive regions of the inflatable member) the position of the ablation elements must be determined only once. The inflated member then anchors the ablation elements in position and the entire procedure can be performed with less movement, in a more stable manner with fewer manipulations by the user. The user locates the lesion, places the inflatable member in the desired position and inflates the member to engage the target tissue. Once the inflatable member has been inflated in the desired position, energy can be applied to the target tissue.

FIG. 1 shows a first exemplary embodiment of an inflatable conductive device 100 according to the invention. The device 100 comprises an inflatable member 102 (e.g., a balloon) which is adapted to be inflated with a fluid introduced, for example, through an inflation duct 114. The inflation fluid may be any fluid suitable for medical uses, such as, for example, saline solution or another inert and biocompatible liquid or gas. The inflation duct 114 is connected at the proximal end to a source of the inflation fluid (not shown). The source may be conventional, and may include a syringe, a fluid container, a compressor, or other device to provide the fluid at an appropriate pressure to inflate the inflatable member 102. The inflatable member 102 may be constructed as described in U.S. patent application Ser. No. 10/663/176 to Greg Eberl and Mark D. Forrest, filed on Sep. 15, 2003, the entire disclosure of which is expressly incorporated herein by reference.

In a deflated, insertion configuration, the inflatable member 102 is wrapped around an elongated member 104 sized to fit through the working channel of an endoscope or similar instrument. Although the inflatable member 102 is shown disposed at a distal end of the elongated member 104, those of skill in the art would understand that the inflatable member 102 may be wrapped around any portion of the elongated member 104. The device 100 is inserted into the working channel of the endoscope which has been positioned in proximity to the target tissue using vision devices of the endoscope, as would be understood in the art. The device 100 is then pushed through working channel until the inflatable member 102 is in a desired position relative to the target tissue (i.e., until the conductive portions of the inflatable member 102 are located adjacent to the target tissue). An inflation fluid is then provided to the inflation duct 114 to inflate the inflatable member 102 to an inflated configuration in which all of the conductive portions thereof are in contact with the target tissue. That is, the inflatable member 102 may be inflated until the entire circumference of each of the bands 110, 112 is in contact with the tissue of the lumen around an entire circumference thereof.

For ablation regions that are to be completely circumferential, the inflatable member 102 will preferably have dimensions allowing inflation to a diameter sufficient to place it in contact with and to press against an entire circumference of the inner walls of a hollow organ containing the target tissue. If, for example, the device 100 is to be used for GERD procedures, the inflated diameter of the inflatable member 102 is preferably substantially similar to, or slightly larger than, an inner diameter of the esophagus. The slightly larger diameter ensures that a stabilizing force will be applied between the esophagus and the inflated inflatable member 102.

The elongated member 104 is connected to the inflatable member 102 to provide structural support and to house the inflation duct 114 and an electrical conductor 116, etc. so that the inflatable member 102 may be pushed through the working lumen of the endoscope to reach the target tissue. Those skilled in the art will understand that a conductive inflation fluid (e.g., saline solution) may serve as the conductor 116 in which case, no separate conductor 116 will be required. In such a situation, a lumen which would otherwise have held the conductor 116 may be eliminated or used for a guidewire or other purpose. The elongated member 104 may also include a proximal handle which allows the inflatable member 102 to be manipulated into the desired position relative to the target tissue. Conventional connections for the inflation duct 114 and the electric conductor 116 may be provided, for example at the proximal end of the elongated member 104 to supply inflation fluid and RF energy to the inflatable member 102. Although, the present invention will be described with respect to the use of RF energy, those of skill in the art would understand that the present invention is not limited to the use of RF energy, but may further or alternatively be used with microwave energy, ultrasonic energy, etc.

According to exemplary embodiments of the present invention, electrical (or RF) energy is applied to selected portions of the target tissue via conductive regions of the inflatable member 102. For example, as shown in FIG. 1, conductive regions 110, 112 may be shaped as circumferential bands with non-conductive regions 108. separating the conductive regions 110, 112. The conductive regions 110, 112 cooperate with the non-conductive regions 108 to ablate two circumferential regions which circumscribe a completely circumferential portion of tissue along a length of the hollow organ extending between the regions 110, 112.

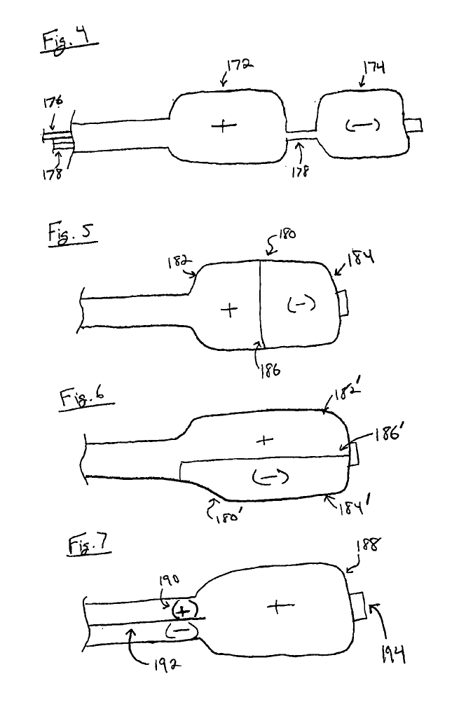

Alternatively, a bi-polar device may include two or more reservoirs of conductive solution electrically insulated from one another. In addition, patterns of conductive and non-conductive regions may be formed on the surface(s) of these reservoirs as described more fully below to obtain desired ablation patterns. For example, as shown in FIG. 4, a bi-polar device may include proximal and distal inflatable members 172, 174, respectively, each of which is inflated with a conductive solution and electrically insulated from the other. In this example, the distal inflatable member 174 is connected to a negative polarity energy while the proximal inflatable member 172 is connected to a positive polarity energy. Separate inflation ducts 176 and 178 are provided for the proximal and distal inflatable members 172, 174, respectively, to maintain their electrical isolation. Those skilled in the art will understand that results similar to those achieved with any bi-polar device may be achieved through the use of 2 or more mono-polar devices.

In a further exemplary embodiment shown in FIG. 5, the bi-polar device may include a single inflatable member 180 divided into first and second reservoirs 182, 184, respectively, each being electrically isolated from the other. As with the above described embodiment, this electrical isolation may be facilitated by providing electrically isolated inflation lumens for the supply of conductive inflation fluid thereto. In this example, the fluid in a first one of the inflation lumens extending to the inflation first reservoir 182 is connected to a source of positive polarity energy while the a second inflation lumen extending to the second reservoir 184 is connected to a source of negative polarity energy. In this embodiment, the first and second reservoirs 182, 184 are separated from one another along the axis of the elongated member by a partition 186 disposed laterally relative to a longitudinal axis of the inflatable member 180. According to an alternate embodiment shown in FIG. 6, a single inflatable member 180′ is separated into first and second reservoirs 182′, 184′, respectively, angularly spaced from one another by a partition 186′ extending along the longitudinal axis of the inflatable member 180′. As shown in FIG. 7, a further exemplary embodiment of the bi-polar device according to the present invention includes a single inflatable member 188 connected to a distal end of each of a first inflation duct 190 for supplying a conductive solution with a first polarity energy (e.g., positive) and a second inflation duct 192 for supplying a conductive solution with a second polarity energy (e.g., negative). As noted above, the first and second inflation ducts 190 and 192 are electrically insulated from each other.

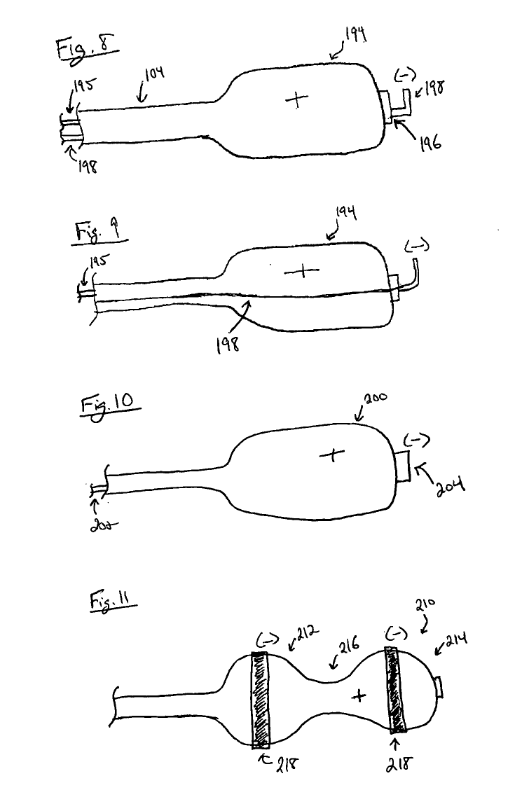

As shown in FIG. 8, an inflatable member 194 is inflated to contact a desired portion of tissue, and a conducting wire 198 is slid through the elongated member 104, the inflatable member 194 and distally out of a distal opening 196 to contact a second spot on the tissue to be ablated. Those of skill in the art will understand that the conducting wire 198 may be replaced by, for example, a guide wire, a conducting ribbon or the like. The conducting wire 198 may be pre-stressed so that, when it exits the distal opening 196, it assumes a curved shape with a distal end thereof facing laterally with respect to the axis of the elongated member 104. Alternatively, the conducting wire 198 may be made steerable using known mechanisms. An inflation duct 195 for inflating the inflatable member 194 is connected to a source of energy of a first polarity (e.g., positive) while energy of the opposite polarity (e.g., negative) is supplied to the conducting wire 198 to ablate the target tissue.

A further exemplary embodiment of the bi-polar device according to the invention is shown in FIG. 9. In this embodiment, a single inflatable member 194 is provided with positive polarity energy via a conductive solution supplied thereto by an inflation duct 196. An electrical conductor 198 (e.g., conducting wire) extends along an outer surface of the elongated member 104 and over an outer surface of the inflatable member 194 to the distal end of the elongated member 104 with all but a distal most portion of the conductor 198 being electrically insulated. In the embodiments of FIGS. 8 and 9, the conductor 198 and the inflatable member 194 may be electrically insulated from each other.

As shown in FIG. 10, a further exemplary embodiment of the bi-polar device according to the present invention includes an inflatable member 200 used to ablate a desired portion of tissue. The inflatable member 200 is provided with a positive polarity energy via a conductive solution supplied thereto by an inflation duct 202. A conductor 204 providing a negative polarity energy may be connected to the bi-polar device, for example, at a distal end of the inflatable member 200. In a further embodiment, the conductor 204 may be disposed at a proximal end of the inflatable member 200, within or outside of the inflation duct 202.

From the disclosure herein, those of skill in the art would understand that the bi-polar device may include more than one inflatable member with opposite polarity energies applied to each. In further embodiments, the bi-polar device may include one or more inflatable members with a first polarity energy applied thereto, and one or more conductors with a second polarity energy applied thereto. In these embodiments, it is preferred that the first polarity energy (e.g., positive) is an opposite of the second polarity energy (e.g., negative). Furthermore, those of skill in the art would understand that it is preferable to maintain a first charged portion (e.g., positive) electrically isolated from a second charged portion (e.g., negative).

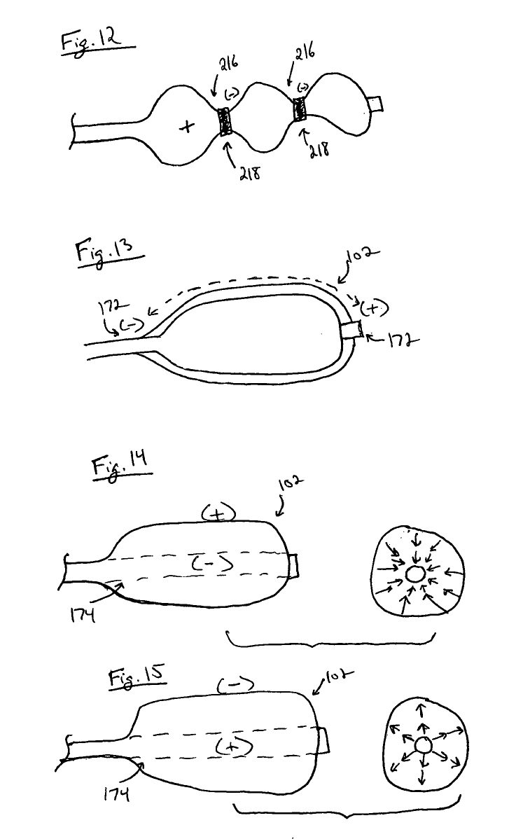

A further exemplary embodiment of the bi-polar device according to the invention is shown in FIGS. 11 and 12. In FIG. 11, an inflatable member 210 includes a first portion 212 and a second portion 214 separated by a reduced cross-section portion 216 therebetween. As understood by those of skill in the art, the inflatable member 210 may be manufactured to inflate to the configuration shown in FIG. 11. Alternatively, the inflatable member 210 may have a uniform cross-section, with a girdle (not shown) wrapped around a portion thereof to restrict expansion and create the reduced cross-section portion 216 when the inflatable member 210 is inflated. In this embodiment, one or more conductive bands 218 are wrapped around the inflatable member 210. In this manner, a conductive solution supplied to the inflatable member 210 energized with a first polarity energy (e.g., positive) and while a second polarity energy (e.g., negative) is supplied to the conductive bands 218. Those of skill in the art will understand that the conductive bands 218 may be formed integrally with the inflatable member 210 or formed of separate members affixed thereto.

As shown in FIG. 12, the conductive bands 218 may alternatively be disposed around the reduced cross-section portion(s) 216. In this embodiment, the conductive bands 218 are recessed from the portion of tissue to be ablated, creating a space therebetween. The space between the tissue and the conductive bands 218 may facilitate an RF application such as, for example, a cutting and/or coagulation waveform.

In one exemplary embodiment, the conductive and non-conductive regions 110, 112 and 108, respectively, of the inflatable member 102 are defined by forming the inflatable member 102 of a conductive polymer (described in more detail below) with non-conductive coatings formed on the regions 108 by, for example, masking. Thus, the regions 108 become non-conductive due to the covering while the regions 110, 112, which are not covered, remain conductive. As would be understood by those skilled in the art, any of various conventional methods may be used to mask the regions 108 of the inflatable member 102 with the non-conductive coating. Alternatively, a balloon may be made entirely of non-conductive material with conductive regions formed through masking with an electrically conductive material.

When the device 100 according to embodiments of the present invention is used to perform an endoscopic mucosal resection, the endoscope is first advanced through a body lumen to a position adjacent to the target tissue. The target tissue is then prepared, for example, by inserting an injection device through the endoscope to subcutaneously inject fluid under the mucosal tissue to “lift” it from the underlying tissue, as would be understood by those skilled in the art. For example, saline solution with or without additional medication such as heparin may be used. After the lifting step has been carried out, the device 100 is inserted through the endoscope to a desired position relative to the target tissue (e.g., under visual control using the optics of the endoscope). Those skilled in the art will understand that other delivery/visualization methods may be used without departing from the scope of the invention. The inflatable member 102 is then inflated until its surface (or at least the conductive portions thereof) is in contact with the inner surface of the lumen. Electrical or RF energy is then applied to the tissue via the conductive regions 110, 112 to ablate the target tissue. Those skilled in the art will understand that this ablation may be controlled to achieve other ends than severing tissue. For example, energy may be applied to stretch tissue, etc.

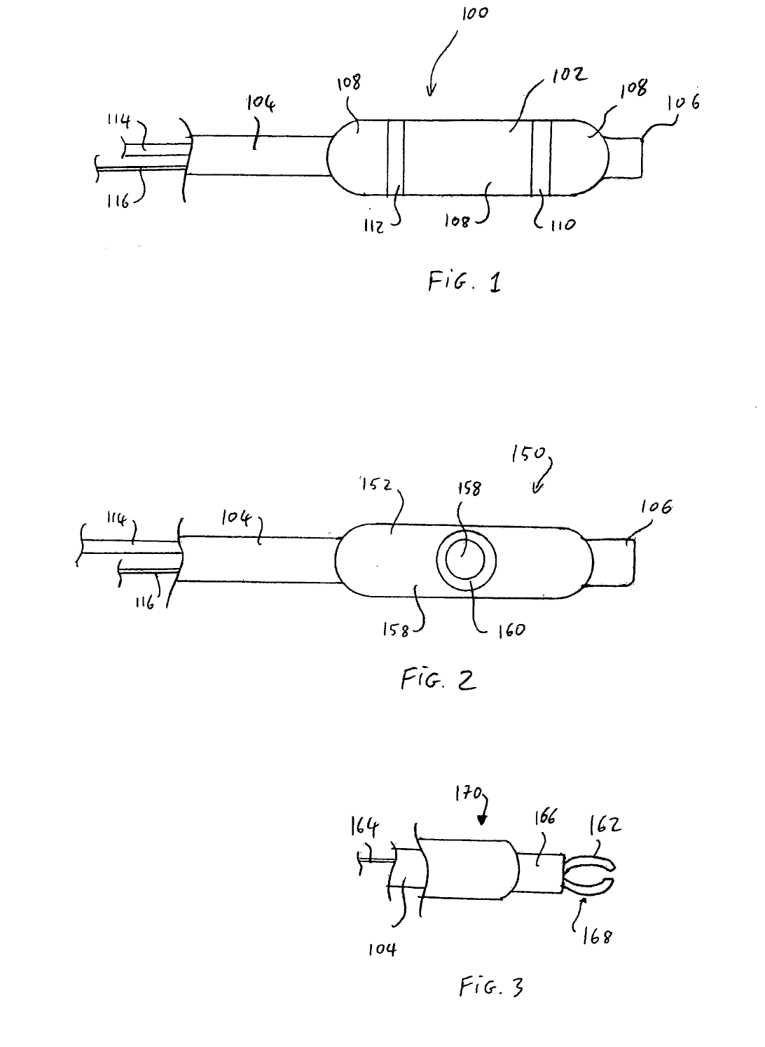

As described above, the pattern of the conductive regions of the inflatable member 102 shown in FIG. 1 creates two circumferential bands of ablated tissue to facilitate, for example, removing a cylindrical section of mucosal tissue previously lifted from the underlying tissue by subcutaneous injection. Of course, different conductive patterns and sizes of inflatable members 102 may be used to remove differently shaped or sized portions of mucosal tissue. For example, as shown in FIG. 2, a substantially circular conductive region 160 may be formed on an inflatable member 152 of a conductive device 150. In this case, regions 158 are non-conductive and do not transfer RF energy to target tissue abutting thereon so that a circular area of target tissue is ablated by this pattern of conductive regions 160, which may be better suited for removal of a localized abnormal lesion of the target tissue. As will be apparent to those of skill in the art, additional patterns and sizes (e.g., elliptical, etc.) of the conductive region(s) of the inflatable members 102, 152 may be devised, to treat a variety of sizes and shapes of regions of target tissue.

Inflatable members for use with the conductive inflation fluids (i.e., those inflatable members where the energy is transmitted via the inflation fluid) according to the present invention are preferably formed of a hydrophilic conductive polymer. Such materials allow electricity to be conducted therethrough via a conductive fluid, such as a saline water solution or other ionic media. An inflatable member formed of a hydrophilic polymer includes free volumes or nanopores through which the inflation fluid permeates to migrate across a surface thereof. In these exemplary embodiments, when the inflatable members are filled with conductive fluid, the degree of conductivity is dependent on the hydrophilic properties of the polymer, the density and porosity of the material, and other parameters which influence the amount of fluid migration therethrough. An exemplary material which may be used to form a conductive inflatable member such as the inflatable members 102, 152 is the Tecophilic 60D-35 material with 35% hydration, manufactured by Thermedics.

In a different embodiment, the conductive inflatable members 102, 152 may be formed from a conductive polymer which conducts electrical (RF) energy without hydration with a conductive fluid. For example, materials such as polyacetylene, polydiacetylene and polyaniline may be used to form the inflatable members 102, 152. These materials may require some manipulation to make them conductive. However, the elimination of the need to hydrate the inflatable member 102, 152 with a conductive inflation fluid simplifies the medical procedure as the inflation fluid may be selected from a larger group of fluids. As with the hydrophilic polymers described above, non-conductive regions may be formed on inflatable members 102, 152 formed of these materials by masking the conductive material with a non-conductive polymer in the desired non-conductive regions. Alternatively, some of the steps necessary to cause the polymers to become conductive may be omitted in the desired non-conductive regions of the inflatable members 102, 152, resulting in a desired pattern of conductive and non-conductive regions. Alternatively, the entire surface of the inflatable members may be made conductive.

As described above, the proximal end of the conductor 116 is connected to a conventional source of RF energy and the distal end is connected to one of the inflatable members 102, 152. Various types of connections between the distal end of the conductor 116 and the conductive inflatable member 102, 152 will be suitable depending on the material and design of the inflatable member 102, 152. When a hydrophillic polymer is used to form the inflatable member 102, 152, the conductor 116 will be exposed to a space into which the conductive fluid will be filled so that, when the inflation fluid is supplied to the inflatable member 102, 152, the distal end of the conductor 116 will be in electrical contact with the conductive inflation fluid. When the inflatable member 102, 152 is formed of a conductive polymer, the conductor 116 may be connected directly to the conductive regions of the inflatable member 102, 152.

FIG. 3 shows a device 170 according to a further exemplary embodiment of the present invention including a device 168 for retrieving the tissue which has been ablatively resected by the device. For example, when energy is applied to the conductive patterns 110, a fully circumferential portion of the mucosal tissue the length of the separation between the patterns 110 and 112 is treated. As would be understood by those skilled in the art, if treatment is continued for a time and at an energy level sufficient to completely ablate the tissue, this central portion of tissue will be resected. The retrieving device 168 shown in FIG. 3 may then be extended from the distal end 166 of the device 170 to grasp this resected tissue. The distal end 166 forms a base or anchor for the grasping device 168 which may be remotely activated via, for example, an actuator coupled to the device 168 by known mechanisms. For example, the grasping arms 162 may be slidable or pivotable with respect to the distal end 166 and may be actuated by an actuator cable 164 connected to an actuator (not shown) which remains outside the body during use of the device 170. Alternatively, the grasping device 168 may be actuated using fluid power, elastic members or electric actuators, as would be understood by those of skill in the art.

Any of the conductive devices according the preceding exemplary embodiments may be used for other medical procedures in addition to the ablation, resection and cauterization operations described above. For example, the conductive elements of the inflatable member 102, 152 may be used to apply a voltage to tissue to achieve electroporation through membranes of the tissue. Electroporation is the physical process of inducing a transient permeability of a biological membrane by the application of pulses of electric voltage of a specified length and strength. When an electric field of appropriate strength and duration is applied to, for example, a cellular membrane through which certain molecules are normally unable to penetrate, aqueous pores are formed through which these molecules may pass to enter the cell. Thus, this process may be induced through the controlled delivery of voltage to transport molecules into and/or out of cells, through the cellular membranes.

Conductive devices according to any of the embodiments of the invention may also be used to induce electrophoresis of biological matter from target tissue into the conductive device or to introduce therapeutic materials included in the inflation fluid into the tissue. Electrophoresis may be used in conjunction with electroporation to extract biological material from a cell through the cellular membrane by applying an electric field to the cell. The electric field causes charged particles to migrate through the cellular membrane. When a hydrophillic material is used to form the inflatable member, the electric current travels through the ionic media (e.g., saline solution) permeating the hydrophillic material. Electroporation may also be induced using a catheter with conductive elements formed directly thereon, rather than on an inflatable member. This arrangement is particularly useful for applications where the target tissue will naturally contact the surface of the catheter (i.e., where a relaxed diameter of the lumen in which the target tissue is located is less than a diameter of the catheter).

According to exemplary embodiments of the invention, the device 100, 150 may be formed such that the electric current flows axially, from one end of the inflatable member 102, 152 to the other end. As shown in FIG. 13, activated electrodes 172 may be placed adjacent the proximal and distal ends of the inflatable member 102, 152. The electrodes 172 may be positioned, for example, similarly to the conductive elements 110, 112 shown in FIG. 1. In one embodiment, the inflatable member 102, 152 may be a single balloon with the conductive solution therein flowing between the electrodes 172. Alternatively, a wall of the balloon may provide a medium for transfer of electric current between the electrodes 172, as shown in FIG. 13. In a further embodiment, two balloons may be provided with the conductive solution injected therebetween. In this embodiment, a first balloon may engulf a second balloon, or, the first balloon may be spaced from the second balloon along the elongated member 104, as shown in FIG. 4.

According to a further exemplary embodiment shown in FIGS. 14 and 15, the flow of current may be directed substantially radially inward (FIG. 14) or outward (FIG. 15) between the surface of the inflatable member 102, 152 and an electrode 174 extending along an internal axis of the inflatable member 102, 152. As understood by those of skill in the art, the electrode 174 may be a conducting wire, ribbon, guide wire, etc.

In any of these exemplary embodiments, biological material will be carried from cells of the target tissue to the interior of the inflatable member 102, 152 where it can be collected and examined to diagnose and/or treat pathologies of the target tissue. Alternatively, the device 100, 150 may include one or more first electrodes on an outer surface of the inflatable member 102, 152 and one or more second electrodes on the elongated shaft 104 to generate a substantially radially directed electric field between the first and second electrodes. Either way, energizing these electrodes with a pulsed voltage temporarily disrupts the membranes of the cells in contact with the inflatable member 102, 152 allowing charged molecules to pass through the cellular membrane to and from the target tissue cells.

As mentioned above, the electroporation which may be induced with the devices 100, 150 according to the present invention may be utilized for therapeutic applications. For example, drugs or biological materials such as DNA may be introduced into cells of the target tissue in this manner. In this procedure, the inflatable member 102, 152 may be filled with a solution including, for example, DNA fragments containing a gene of interest or other therapeutic materials. As described above, the inflatable member 102, 152 is placed in a desired position with respect to the target tissue and inflated to contact the target tissue. Pulses of DC current are then supplied to the conductor 116 to apply a pulsed electric field to the target tissue via the conductive regions 110, 112 or 160 of the inflatable member 102 or 150, respectively. The pulsed electric field temporarily disrupts the membranes of the target cells and permits the DNA solution or other therapeutic material to migrate across the cell membranes into the cells of the target tissue to treat the cells of the target tissue as would be understood by those skilled in the art. In a further embodiment, the inflatable member 102, 152 may include micropores for leaking the contents therein (e.g., saline, diagnostic agents, therapeutic agents, biological material, etc.) to the target tissue. Those of skill in the art would understand that leaking, for example, saline, may facilitate electroporation, electrophoresis, iontophoresis and/or hydrothermal treatment.

A conductive device according to yet another exemplary embodiment of the present invention may be constructed substantially in accord with any of the above-described embodiments except that, in place of the electrically conductive and insulated regions, the inflatable member according to this embodiment comprises first regions conductive to heat and second regions which are thermally insulated. The thermally insulated regions of the inflatable member's surface may be formed from or coated with a thermally insulative material which, when a heated inflation fluid is introduced into the inflatable member maintains an outer surface temperature cooler than in the thermally conductive regions. Thus, target tissue placed in contact with the thermally conductive regions is ablated while the surrounding tissue in contact with the thermally insulated regions is left intact. As would be understood by those skilled in the art, the heat source used to heat the conductive elements of the device may be an electric heater or any other suitable source of heat compatible with the medical environment. The resulting treatment of target tissue is similar to that obtained when using RF energy. As would be understood, thermally insulated portions of the outer surface may be created by introducing a layer of air between an inner fluid contacting surface of the inflatable member and an outer tissue contacting surface (i.e., by creating air pouches in the areas to be insulated). Alternatively, as would be understood by those skilled in the art, a thickness of the material forming the inflatable member may be increased in the regions to be insulated or a coating may be applied to the areas to be insulated.

An exemplary procedure for using a conductive device according to the present invention is presented below. Although, the device 100 of FIG. 1 will be referenced to describe the procedure, the device 150 shown in FIG. 2 or other conductive devices may be used as well. This exemplary procedure uses a device 100 to treat GI tract mucosal tissue in an endoscopic mucosal resection operation. In a preliminary step, a device 100 is selected having conductive regions 110, 112 which present a conductive surface of a desired size and shape relative to the target tissue to be treated. The device 100 is advanced using the elongated shaft 104 to the location of the target tissue through an endoscope or other similar instrument which preferably incorporates a vision device. Once the device 100 has been placed at the desired location, an inflation fluid is supplied to the inflatable member 102 through the inflation conduit 114 causing the inflatable member 102 to expand until the conductive regions 110, 112 thereof contact the target tissue.

As described above, prior to the resection step, a subcutaneous injection may be used to lift the mucosal tissue from the underlying muscularis propria tissues. For example, saline solution may be injected under the target mucosal tissue, to form a space between the targeted mucosal tissue and muscularis propria tissues below. In this manner, when the target mucosal tissue is severed from surrounding regions of mucosal tissue, it can be removed from the GI relatively easily. It will be apparent to those of skill in the art that other conventional procedures may be used to separate the target mucosal tissue from the underlying tissues.

After the targeted mucosal tissue has been separated from the underlying muscularis, RF energy is provided to the conductive regions 110, 112 through the conductor 116, so that RF ablation of the target tissue takes place along the pattern of the conductive regions 110, 112. In the exemplary embodiment of FIG. 1, the ablation regions encompass two circular bands, so that a tube-like portion of the mucosal tissue in the esophagus is detached from its underlying muscularis propria tissue. The vision system of the endoscope, or other conventional methods, may be used to determine when the RF energy has sufficiently ablated the target mucosal tissue that the treatment may be terminated (i.e., when the target tissue has been sufficiently ablated that adjacent regions of tissue may be easily separated from one another).

After the ablation of the target tissue has been completed, the resected portion of the mucosal tissue may be removed using conventional methods. For example, the device 100 may be withdrawn from the endoscope and a grasping device may be introduced to the target region. Alternatively, the device 100 may comprise a grasping device such as the grasping device 168 shown in FIG. 3. In this exemplary embodiment the removal of the resected portion of the target mucosal tissue is simplified, because the conductive device 170 may be withdrawn at the same time as the resected target tissue, without the need for additional steps.

As described above, alternatively, medication may be provided to the target tissue through electroporation by applying an electric field to the target tissue through the conductive elements 110, 112, 160 or similar elements at a level low enough or at a frequency selected so that the tissue is not ablated. Thus, therapeutic compounds included in the inflation fluid are introduced into the cells of the target tissue through the cellular membranes. As indicated above, this procedure is well suited for genetic therapy to treat the target tissue.

As would be understood by those skilled in the art, a degree of ablation of the target tissue may be determined by measuring an impedence of the tissue. Feedback control may then be used to stop the supply of energy when a desired level of ablation has been achieved.

The present invention has been described with reference to specific exemplary embodiments. Those skilled in the art will understand that changes may be made in details, particularly in matters of shape, size, material and arrangement of parts without departing from the teaching of the invention. For example, the conductive device may be optimized to resect, cauterize and ablate different organs in addition to components of the GI tract. Furthermore, those skilled in the art will understand that the above-described apparatus and procedures may be enhanced if combined with tools including knives, graspers, suction, vibration , etc. Accordingly, various modifications and changes may be made to the embodiments without departing from the broadest scope of the invention as set forth in the claims that follow. The specifications and drawings are, therefore, to be regarded in an illustrative rather than a restrictive sense.

What is claimed is:

1. A tissue treatment device comprising:
an elongate member including a lumen extending therethrough to an opening formed in a distal portion thereof;
an inflatable member mounted to the distal portion of the elongated member and surrounding the opening, an outer surface of the inflatable member including a conductive region having a first degree of energy conductivity and a non-conductive region having a second degree of energy conductivity, the second degree of energy conductivity being lower than the first degree of energy conductivity; and
an energy source for supplying energy to the inflatable member to transfer energy from the conductive region to target tissue.

2. The tissue treatment device according to claim 1, wherein the elongated member is laterally flexible but has a column strength sufficient to allow the elongated member to be advanced through a flexible endoscope to a target site within a body.

3. The tissue treatment device according to claim 1, further comprising a tissue grasping mechanism extendible from the distal portion of the elongated member.

4. The tissue treatment device according to claim 3, further comprising an actuator mounted to a proximal end of the elongated member, the actuator being coupled to the grasping mechanism so that actuation of the actuator operates the grasping mechanism.

5. The tissue treatment device according to claim 1, wherein the conductive region conducts one of heat and electrical energy.

6. The tissue treatment device according to claim 1, wherein the inflatable member is formed of a hydrophilic polymer.

7. The conductive inflatable device according to claim 6, further comprising a source of inflation fluid selectively coupleable to the lumen for supplying inflation fluid to the inflatable member, wherein the inflation fluid is an ionic media.

8. The tissue treatment device according to claim 1, wherein the inflatable member is formed of an electrically conductive polymer.

9. The tissue treatment device according to claim 8, wherein the conductive polymer is one of polyacetylene, polydiacetylene and polyaniline.

10. The tissue treatment device according to claim 1, wherein the non-conductive region comprises an electrically insulative conductive polymer.

11. The tissue treatment device according to claim 10, wherein the non-conductive region is formed by one of coating, layering and cladding of the non-conductive polymer over an electrically conductive material which forms the inflatable member.

12. The tissue treatment device according to claim 1, wherein the conductive region comprises a plurality of discreet portions of the outer surface of the inflatable member.

13. The tissue treatment device according to claim 1, wherein the first and second degrees of energy conductivity are degrees of heat conductivity.

14. The tissue treatment device according to claim 13, wherein the source of energy comprises a heater for heating the inflation fluid.

15. The tissue treatment device according to claim 1, wherein the first and second degrees of energy conductivity are degrees of electrical conductivity.

16. The tissue treatment device according to claim 15, further comprising a source of inflation fluid selectively coupleable to the lumen for supplying inflation fluid to the inflatable member, wherein the source of energy comprises a source of electrical energy coupled to an electrode in electrical contact with a space within the inflatable member into which the inflation fluid will be supplied to inflate the inflatable member.

17. The tissue treatment device according to claim 16, wherein the inflatable member is formed of an electrically conductive polymer and wherein the source of energy comprises a source of electrical energy coupled to the inflatable member.

18. The tissue treatment device according to claim 1, wherein the inflatable member has inflated dimensions selected so that the conductive region contacts the target tissue.

19. The tissue treatment device according to claim 18, wherein the inflated dimensions are adapted to place the conductive region in contact with an inner surface of a hollow organ.

20. The tissue treatment device according to claim 1, wherein the conductive region defines a selected ablation pattern.

21. The tissue treatment device according to claim 22, wherein the conductive region defines one of a circular and an oval conductive pattern circumscribing a portion of the non-conductive region.

22. The tissue treatment device according to claim 22, wherein the inflatable member extends along a longitudinal axis of the elongated member and wherein the conductive region defines a first annular ring which, when inflated in contact with target tissue within a hollow organ, extends around a circumference of the organ.

23. The tissue treatment device according to claim 24, wherein the conductive region defines a second annular ring which, when inflated in contact with target tissue within a hollow organ, extends around a circumference of the organ, the first and second annular rings circumscribing a longitudinal portion of an inner surface of the hollow organ.

24. A method of treating tissue comprising:
inserting an elongated member including an inflatable member mounted thereto through a body lumen to a desired position therein, the inflatable member including a conductive region having a first degree of energy conductivity and a non-conductive region having a second degree of energy conductivity lower than the first degree of energy conductivity;
inflating the inflatable member to place the conductive region in contact with a target portion of tissue; and
supplying energy to the inflatable member to transfer energy from the conductive region to the target tissue.

25. The method according to claim 24, wherein the target portion of tissue is mucosal tissue further comprising lifting the target mucosal tissue from underlying tissue, wherein the energy supplied to the target mucosal tissue separates the target tissue from surrounding portions of tissue.

26. The method according to claim 25, wherein the conductive region circumscribes a portion of tissue to be removed and wherein an amount and intensity of the energy supplied to the target tissue is selected to ablate the target tissue to sever the circumscribed tissue from surrounding tissue, further comprising employing a removal device to remove the circumscribed tissue from the body.

27. The method according to claim 26, wherein the conductive region comprises circumferential annular bands.
